(19) United States
(12) Patent Application Publication
(10) Pub. No.: US 2017/0017767 A1
(43) Pub. Date: Jan. 19, 2017

(54) PATIENT WATCH-DOG AND INTERVENTION/EVENT TIMELINE

(71) Applicant: KONINKLIJKE PHILIPS N.V., EINDHOVEN (NL)

(72) Inventors: Abigail Acton FLOWER, MAHOPAC, NY (US); Sophia Huai ZHOU, CAMBRIDGE, MA (US)

(73) Assignee: Koninklijke Philips N.V., Eindhoven (NL)

(21) Appl. No.: 15/123,690

(22) PCT Filed: Mar. 13, 2015
(86) PCT No.: PCT/IB2015/051861

§ 371 Date: Sep. 6, 2016

Related U.S. Application Data
(60) Provisional application No. 61/952,159, filed on Mar. 13, 2014.

(51) Int. Cl. G06F 19/00; A61B 5/00; A61B 5/0205

(52) U.S. Cl. CPC G06F 19/3418; G06F 19/322; G06F 19/3431; A61B 5/0205; A61B 5/7425; A61B 5/743; A61B 5/021

(57) ABSTRACT

A medical system includes a plurality of portable vital sign monitors (12), a patient assessment unit (24), a patient watch-dog unit (42), and at least one display device (44). Each monitor continuously monitors vital signs of a subject (12) from a point of initial contact with a healthcare professional and wirelessly transmits the monitored vital signs, and the vital signs include blood pressure (BP), blood oxygen (SpO2), heart rate (HR), and respiratory rate (RR). The patient assessment unit (24) receives the transmitted monitored vital signs of each subject and determines a triage score based on the received vital signs, and subject gender, subject age, and subject symptoms. The patient watch-dog unit (42) constructs a display of a patient trajectory of each subject and the trajectory includes the monitored vital signs, the determined triage score, and the subject gender, the subject age, and the subject symptoms. The at least one display device (44) display the constructed patient trajectory of at least one subject.

The following relates generally to patient monitoring. It finds particular application in conjunction with patient triage assessment and continuous patient monitoring, and will be described with particular reference thereto. However, it will be understood that it also finds application in other usage scenarios and is not necessarily limited to the aforementioned application.

Hospital emergency departments are often an initial point of contact between persons or emergency patients and the healthcare system. Patients with emergency medical conditions encounter the emergency department with a wide variety of undifferentiated medical, surgical, psychological, and social problems. Doctor's offices which can see patients with undifferentiated problems encourage patients with an emergency to go directly to a hospital emergency department (ED) or emergency room. Patient entering other hospital departments typically have differentiated problems specific to the services of the entered hospital department and are generally triaged by a nurse based on the patient symptom and conditions, patients with less severe symptom and conditions are treated on a first come first serve basis. The point of initial contact for emergency departments is typically a triage healthcare professional.

The triage healthcare professional performs an initial assessment of an arriving emergency patient, and assigns a triage score. The initial assessment can include measures of vital signs of the arriving emergency patient. The triage score can be numeric and/or color coded. The triage score is a measure of the severity of the emergency and the immediacy of access to healthcare resources needed. For example, a score of 1 or black can indicate so severe injuries that the subject is unlikely to survive; a score of 2 or red can indicate required immediate surgery or other life-saving intervention; a score of 3 or yellow can indicate a stable condition but requires hospital care; 4 or green can indicate a doctor's care is needed, but can treatment can wait; and a score of 5 or white can indicate minor injuries for which first aid or other non-emergency treatment may be sufficient. Different scoring systems can and are used by different healthcare providers. Emergency patients are admitted to the ED and treatment based on the severity and immediacy of access needed. Emergency patients not immediately admitted to the ED wait, typically in a waiting room or waiting area. As emergency patients are treated in the ED and their conditions differentiated, the patient is either transferred to an appropriate treatment unit of the hospital or discharged. Waiting emergency patients are admitted according to their triage score. As new emergency patients arrive, they are assessed and assigned a score and either admitted to the ED or the waiting room. Triage prioritizes access to limited resources of healthcare. The limited resources can be any one of bed space, personnel, or other resource constraint, or a combination.

Visits to hospital emergency departments are increasing while the number of emergency departments is decreasing which contributes to increased wait times. Patient visits to the emergency department increased an estimated 32% between 1999 and 2009. The increase in visits is associated with longer wait times for ED patients, particularly in urban areas. A 2012 California hospital review demonstrated an association between an increased waiting time and a 5% increase in patient mortality. Emergency patient conditions can and do change from the time of the triage assessment to the time the emergency patient is admitted to the ED.

An ED healthcare professional often faces a difficult task of differentiating problems with little data about the immediate emergency, and time constraints to access potential voluminous amounts of data which may or may not be relevant to the problems. For example, the admitted ED patient may be accompanied by a chart which shows the vital signs taken at the triage assessment, and the complaint presented at that time. That may be followed by hours of wait time and a change in condition by the time the ED patient is admitted. The ED patient may not even be conscious anymore by an event that triggered the ED admission from the waiting area, e.g. collapsing in the waiting room or hallway. The ED healthcare professional has little time and little information to develop an understanding of the current situation which may involve stabilizing the ED patient, e.g. how long has the patient been like this, is the condition worsening, etc.

Additionally, patient data can be spread across multiple systems and systems which are structured for general hospital use such as electronic medical records (EMR), Picture Archiving and Communication System (PACS), Admit/Discharge/Transfer (ADT) system, and the like. The healthcare practitioner navigates across each system to search and obtain relevant information. Relevant information can include prior vital signs, diagnoses, therapies, and the like. The healthcare practitioner must then identify trends based on retrieved relevant information.

Transitioning patients from one unit to another typically involves transitioning from one system to another. For example, as a patient is transferred from the ED to another unit in the hospital, the monitoring and record keeping system may change from the ED system to a specialty unit or other departmental system. The transition can include a change in scoring systems such as a change from the triage score to a Sequential Organ Failure Assessment (SOFA) score or other scoring system. Healthcare practitioners in the specialty unit or other department typically begin again to understand the current condition of the patient and build an understanding of the events leading to admission to the particular unit.

There are typically multiple individuals in different roles working as a team with many ED patients, doctor, nurse, technician, etc. Communication of the situation of each patient includes reviewing data from different sources, triage assessment, recorded records and systems, and developing a coordinated plan of action. How the information is communicated about each patient in a team environment can affect outcomes in terms of time and in terms of quality of the communication, e.g. communicating the necessary information at the right time in an environment where the communication is voluminous. Too many communications, and the important communications can be overwhelmed and lost. Too little communication, and the needed information might not be communicated to the responsible healthcare professional. For example, a doctor orders a therapy such as a medication for a patient and the time is typically recorded in the medical record for which the doctor must check back to assess the expected change in condition based on the actual time of administration and not the time ordered. The communication includes writing the order, receiving the order by one who obtains the medication, administering the medical to the patient, recording the time in the chart of administration, checking the time of administration, and assessing the change in condition. If a notice is sent each time a step is performed, the communication system of a healthcare practitioner such as a smartphone, pager, etc., can be flooded with notices. If no communication is made and the healthcare practitioner is busy with another patient, the change or lack of change in condition may not be promptly addressed.

The following discloses a new and improved patient watch-dog and intervention/event timeline system which addresses the above referenced issues, and others.

In accordance with one aspect, a medical system includes a plurality of portable vital sign monitors, a patient assessment unit, a patient watch-dog unit, and at least one display device. Each monitor continuously monitors vital signs of a subject from a point of initial contact with a healthcare professional and wirelessly transmits the monitored vital signs, and the vital signs include blood pressure (BP), blood oxygen (SpO2), heart rate (HR), and respiratory rate (RR). The patient assessment unit receives the transmitted monitored vital signs of each subject and determines a triage score based on the received vital signs, and subject gender, subject age, and subject symptoms. The patient watch-dog unit constructs a display of a patient trajectory of each subject and the trajectory includes the monitored vital signs, the determined triage score, and the subject gender, the subject age, and the subject symptoms. At least one display device displays the constructed patient trajectory of at least one subject.

In accordance with another aspect, a method of monitoring patients includes receiving continuously monitored and transmitted vital signs of a plurality of subjects, each subject monitored from a point of initial contact with a healthcare professional and the vital signs include blood pressure (BP), blood oxygen (SpO2), heart rate (HR), and respiratory rate (RR). A triage score is determined for each subject based on the received vital signs, subject gender, subject age, and subject symptoms. A display of a patient trajectory is constructed for each subject and the trajectory includes the monitored vital signs, the determined triage score, and the subject gender, the subject age, and the subject symptoms. The constructed patient trajectory of at least one subject is displayed on a display device.

In accordance with another aspect, a medical system includes a plurality of portable vital sign monitors, a patient assessment unit, an event unit, and a patient watch-dog unit. Each monitor continuously monitors vital signs of a subject from a point of initial contact with a healthcare professional and wirelessly transmits the monitored vital signs. The patient assessment unit receives the monitored vital signs of each subject and determines a triage score and at least one sequential organ failure assessment (SOFA) score based on the received vital signs, and recorded patient information. The event unit (32) generates an event based on at least one of a change in the triage score or the SOFA score, a change in a care type, or an administration of a therapy. The patient watch-dog unit constructs a display of a care status display of monitored subjects organized by care type which include a waiting area and an emergency department, and each subject in the waiting area is indicated with a color coded icon indicative of the determined triage score, and each subject in the emergency room is indicated with a color coded icon indicative of a worst determined SOFA score.

One advantage is semi-automated triage for faster and most consistent triage.

Another advantage resides in alerting of a change in condition from the point of first contact with the healthcare system.

Another advantage resides in continuously assessing and reporting patient acuity.

Another advantage resides in the multiple scoring systems and the transition between scoring systems.

Another advantage resides in differentiated communications based on role.

Another advantage includes capture and display of critical events and intervention procedures of each patient.

Another advantage resides in the ability to communicate a patient trajectory.

Another advantage resides in the ability to transition and continue patient monitoring from the point of first contract and departmental transitions until discharge.

Still further advantages will be appreciated to those of ordinary skill in the art upon reading and understanding the following detailed description.

The invention may take form in various components and arrangements of components, and in various steps and arrangement of steps. The drawings are only for purposes of illustrating the preferred embodiments and are not to be construed as limiting the invention.

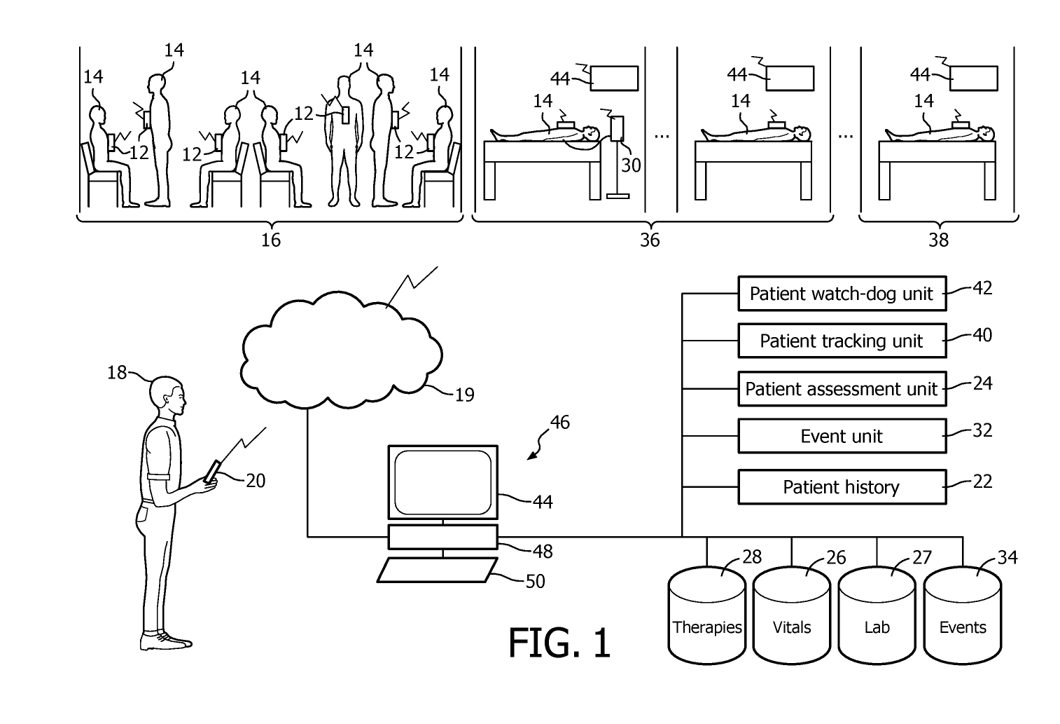

FIG. 1 schematically illustrates an embodiment of a patient watch-dog and intervention/event timeline system.

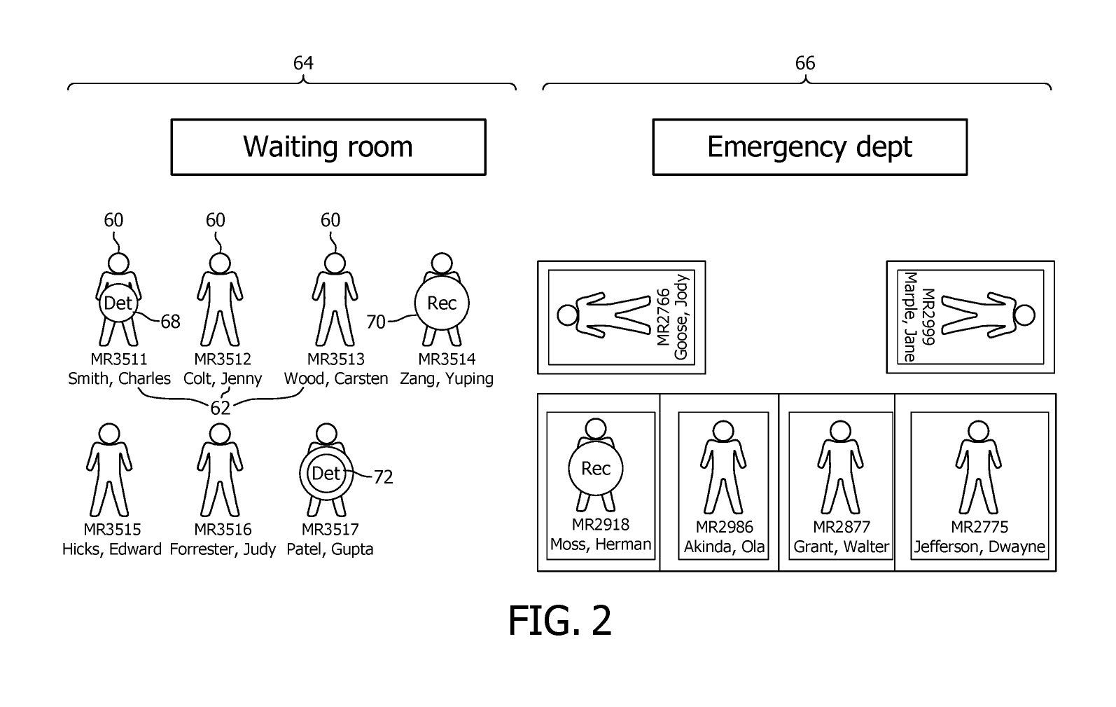

FIG. 2 illustrates an exemplary of a waiting room and an ED display.

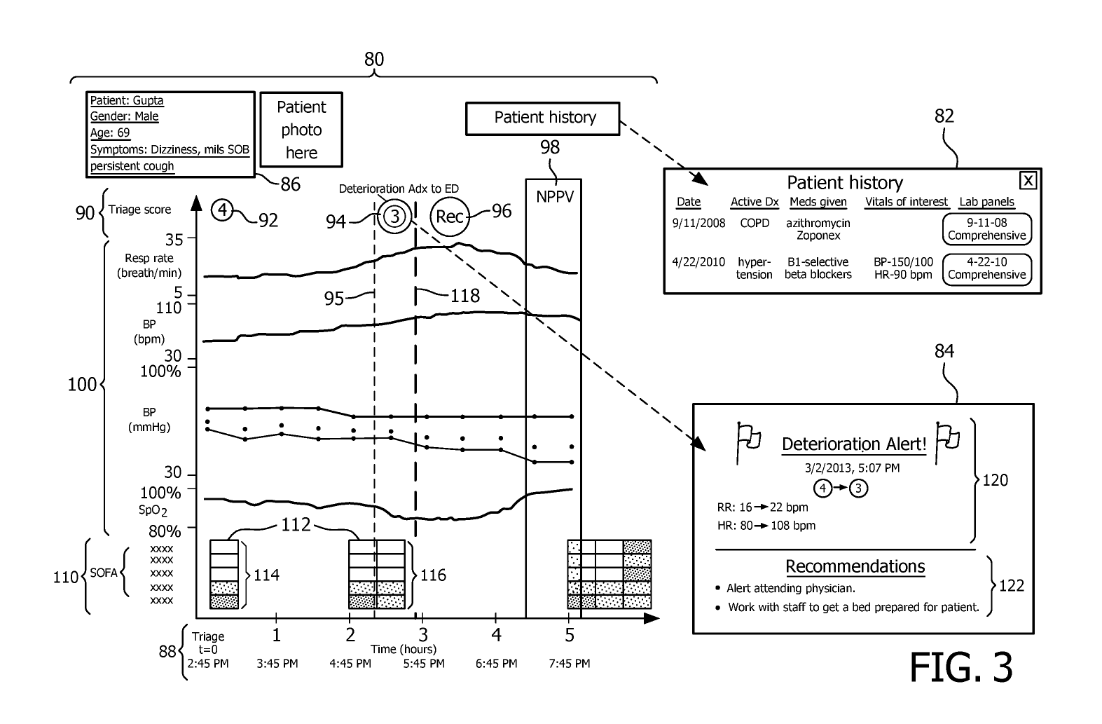

FIG. 3 illustrates an exemplary of a patient watch-dog and intervention/event timeline system dashboard display.

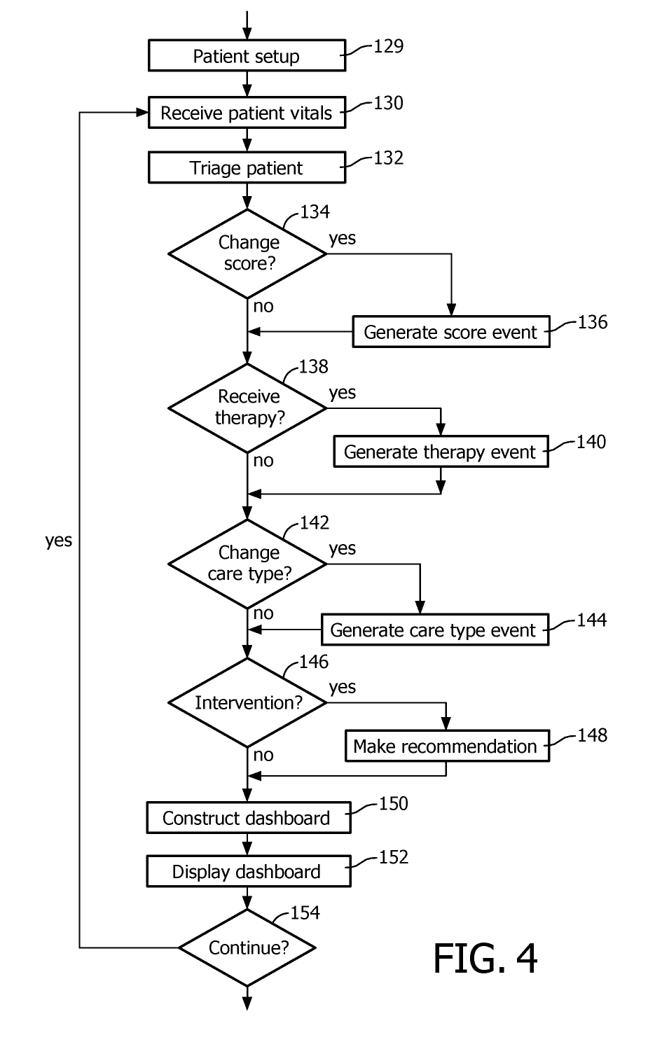

FIG. 4 flowcharts one method of using an embodiment of the patient watch-dog and intervention/event timeline.

With reference to FIG. 1, an embodiment of a patient watch-dog and intervention/event timeline system 10 is schematically illustrated. The system includes a plurality of portable or wearable vital sign monitors 12. Each portable vital sign monitor 12 is attached to a subject 14 such as an emergency patient waiting in an emergency department waiting area 16. A healthcare professional 18 such as a triage nurse attaches each portable vital sign monitor 12 to the subject as each subject arrives and an initial contact is made between the arriving emergency patient and the healthcare professional. Each monitor 12 continuously monitors vital signs of a subject from the point of initial contact with a healthcare professional. Each monitor 12 wirelessly transmits the monitored vital signs. The vital signs transmitted include blood pressure (BP), blood oxygen (SpO2), heart rate (HR), and respiratory rate (RR). The monitor can be a single device or multiple devices. For example, one device can sense BP, SpO2, and HR while an accelerometer can sense RR. The monitors transmit wirelessly over a communications network 19 such as an 802.X wireless network, Blue-tooth™, and the like. The network can be private and/or public, e.g. Internet, cellular or data based, include radio frequency and/or optical communications.

The triage nurse collects initial patient information such as name, age, gender, and symptoms. The collected initial information can be entered via a computing device 20 and/or retrieved by a patient history unit 22 from a patient history data store such as an EMR.

A patient assessment unit 24 receives the transmitted monitored vital signs of each subject and determines a triage score based on the received vital signs, and subject gender, subject age, and subject symptoms. The patient assessment unit 24 can store the received vital signs in a vital sign data store 26. The vital sign data store can be further populated by the patient history unit 22 with prior or historical vital signs, e.g. prior to arrival at the emergency department, but active diagnoses. The patient history unit can link to the electronic medical patient records database of the hospital or hospital system associated with the ED and prior medical history retrieved if the patient has been treated by a clinician in the hospital system. Alternatively, the patient history unit can communicate via any of various electronic systems with a hospital system patient records database to retrieve the patient history information. The assessment unit determines a SOFA score based on the received vital signs and any received lab results. Lab results can be entered or retrieved from a lab data store 27. The assessment operates to continuously receive monitored vital signs and lab results and recomputed the triage and/or SOFA scores. For example, an initial triage score based on the monitored vital signs is determined to be a 4, and later while in the waiting area RR and HR increase rapidly. The assessment unit revises the triage score from a 4 to a 3 based on the increase in RR and HR vital signs. Lab results can be received at any point in time and/or retrieved from lab data store 27, the patient history and/or lab reporting system as the information becomes available.

The patient assessment unit 24 can assess the effectiveness of the administered therapy based on the monitored vital signs and the start of a therapy. For example, a medication can be administered to the subject and recorded in a therapy data store 28 either by entry of the healthcare practitioner or by a therapy delivery device 30. Delivery of a bolus by a medication delivery device is well known in the art. The start of the bolus is recorded.

The assessment unit can further assess trends in the vital signs and adjust a frequency of transmitted vital signs based on the determined triage score. The frequency can be adjusted for each portable monitoring device 12. For example, a score of 4 or 5 can be transmitted less frequent, than a score of 2 which can be transmitted continuously. Frequency of transmission can be a static parameter such as a site based parameter and/or dynamically adjusted such as transmission traffic dependent.

An event unit 32 generates an event based on a change in the triage score or the SOFA score, a change in a care type, an administration of a therapy, and/or an predetermined assessment interval from the therapy administration. The generated event can be recorded in an event data store 34. The generated event can include a recommendation for a therapy and/or a notice. The recommendation can include differentiated notices by role type. For example, a change in triage score can generate an event which includes deterioration notice and a recommendation to an attending physician to consider a chest x-ray and ventilation support.

The change in the care type includes admitting to an ED 36 or to a specialty care unit 38 such as hypertension unit, operation room (OR), post-anesthetic care unit (PACU), cardiac care unit (CCU), intensive care unit (ICU), neuro-ICU, pulmonary ICU, and the like. The change in care type can be recorded by the healthcare professional 18 and/or recorded based on a change in physical location of the subject by a patient tracking unit 40. The tracking is continued following the patient from one department to another department such as from ED to hospital discharge. This tracking can also be done automatically. For example, patient tracking can include the attachment of a wristband with a Radio Frequency IDentification (RFID) tag. The location of the subject can be traced by readers of the RFID placed at doorways or hallways throughout a building. As the subject moves from the waiting area 16 to the ED 36, the patient tracking unit can change the care type to ED. The event unit 32 generates the event for the change to the ED care type.

A patient watch-dog unit 42 constructs a dashboard display of a patient trajectory of each subject. The trajectory includes the monitored vital signs, the determined triage score, and the subject gender, the subject age, and the subject symptoms. The constructed display can include the SOFA scores and the events. The trajectory communicates the patient situation of the subject visually. The trajectory includes provides a perspective of time from the point of initial contact with the healthcare practitioner. The trajectory shows a seamless picture of the health of the patient which can be improving, deteriorating, or stable and in what timeframe and under what care type. The trajectory can include finer distinctions based on the scoring, events, and vital signs. The monitored vital signs constructed display displays graphically the vital signs from the point of initial contact to a current time. The display can be modified to display graphically the vital signs from a particular event forward, e.g. folded at an event. Events and scores can include icon representations. Events, scores, and/or vital signs can be color coded which can communicate different levels of urgency, concern and/or change. As the subject transitions from one care type to another, the appropriate scoring system can be substituted. For example, an ED patient for which a triage score and a SOFA score is displayed is transferred to a cardiac care unit. The display of the SOFA score can be carried forward in the display and a cardiac scoring system substituted for the triage score demarcated by the care type change event.

A display device 44 displays the constructed patient trajectory of at least one subject. The display device can be located in an individual ED room or bedside, as part of a workstation 46 such as a nursing station and/or the computing device 20 which can include a smartphone, tablet, portable computing device, and the like. A ‘display’ or ‘display device’ as used herein encompasses an output device or a user interface adapted for displaying images or data. A display may output visual, audio, and/or tactile data. Examples of a display include, but are not limited to: a computer monitor, a television screen, a touch screen, tactile electronic display, Cathode ray tube (CRT), Storage tube, Bistable display, Electronic paper, Vector display, Flat panel display, Vacuum fluorescent display (VF), Light-emitting diode (LED) displays, Electroluminescent display (ELD), Plasma display panels (PDP), Liquid crystal display (LCD), Organic light-emitting diode displays (OLED), a projector, Head-mounted display, and the like. The display can be automatically triggered by the proximity of the healthcare practitioner device 20 to the patient location.

The workstation 46 includes an electronic processor or electronic processing device 48, the display 44 which displays the constructed display of at least one subject or a composite display of a plurality of subjects, menus, panels, and user controls, and at least one input device 50 which inputs the healthcare practitioner selections. The workstation 46 can be a desktop computer, a laptop, a tablet, a mobile computing device, a smartphone, and the like. The input device can be a keyboard, a mouse, a microphone, and the like.

The various units 22, 24, 32, 40, 44 are suitably embodied by an electronic data processing device, such as the electronic processor or electronic processing device 48 of the workstation 46, or by a network-based server computer operatively connected with the workstation 46 by the network 19, or so forth. Moreover, the disclosed monitoring, patient trajectory and scoring, patient tracking, assessment, event generation, and storage and retrieval techniques are suitably implemented using a non-transitory storage medium storing instructions (e.g., software) readable by an electronic data processing device and executable by the electronic data processing device to perform the disclosed patient monitoring, patient trajectory and scoring, patient tracking, assessment, event generation, and storage and retrieval techniques.

Each of the data stores 26, 27, 28, 34 described herein, can include structured and unstructured data, magnetic and/or optical medium, files systems and/or database organizations, one or more configured processors, one or more servers, and the like.

In FIG. 2 an exemplary display of waiting room and ED is illustrated. Each subject 14 is represented by an icon 60 such as a subject outline which is color coded. Each subject icon 60 includes basic identity information 62 in a text format such as name and/or RFID. The subject icons are organized by care type and/or location which include the waiting room 64 and the ED 66. The waiting room icons are arranged to facilitate reading and can be unordered, or ordered by triage score, name, and/or wait time. The icons 60 in the ED 66 are further arranged by location such as the ED bed location, SOFA score, name, time from last event, etc. The subjects in the waiting room are color coded for the triage score while the subjects in the ED are color coded for the acuity of the worst SOFA score. For example, green for higher triage scores such as a 4 or 5, yellow for intermediate such as a 3 and red for lowest or most acute such as a 1 or 2. The color coding and arrangement facilitate a determination of which subjects can be discharged or moved from the ED (coded green) and which subjects in the waiting room (coded red) need to be admitted into the ED and to which bed. An open bed can be indicated without color, e.g. gray or not green, yellow, or red. Facilitating movement by either identifying and prioritizing usage, or by identifying bed openings in the ED and moving subjects more quickly into bed spaces can reduce the wait times or at least the wait times for the most urgent cases.

The subject icons 60 can be modified with a deterioration icon 68, a recommendation icon 70, or a combination icon 72. The icons are labeled color coded circles superimposed on the subject icons such as red for urgent, gray for not urgent. The deterioration icon 68 indicates a deterioration in the subject condition. The healthcare practitioner can select the icon and receive in a pop-up window the details which contribute to the deterioration status. A recommendation icon can be selected to receive a recommendation which can be further tailored to the healthcare practitioner, e.g. within the scope of duties. The deterioration icon and recommendation icon are different sizes which can be superimposed to form a combination icon 72. The recommendation icon is shown as a larger circle for which the deterioration details or the recommendation can be selected by the selecting the appropriate area of the icon.

In FIG. 3 an exemplary of a patient watch-dog and intervention/event timeline system dashboard display 80 is illustrated with an exemplary patient history popup window 82 and an exemplary deterioration detail and recommendation popup window 84. The dashboard display includes summary patient information 86 located in the upper left hand corner such as name, gender, and age and the symptoms which brought the subject to the ED. The display for a particular subject can be invoked by selecting a subject icon in the display described in reference to FIG. 2.

The patient trajectory is a visual display with minimal textual information. The visual display is ordered by a timeline 88 which begins at the point of initial contact or t=0. The timeline can be indicated in both absolute time, e.g. wall clock time and relative time, e.g. time in hours since initial contact/initial triage assessment. Triage scoring 90 is shown below the summary patient information 86. A triage score icon 92 includes a color coded circle icon with the triage numeric score labeled. The color coding is the same as described in reference to FIG. 2. In the display the triage score is initially 4. At time t=2 hours 20 min, a deterioration in the subject occurs and a combination icon 94 with the changed triage score labeled is displayed. The change in triage score and associated generated event 95 is visually indicated with a dotted line which extends from event time on the timeline to the triage scoring area.

Once the subject is admitted to the ED, the triage scoring space is used for deterioration, recommendation, and/or combinations icons and/or therapy identification 98. At time t=2 hours 54 min, the subject is admitted to the ED and a care type change event is indicated with a differently coded dotted line extending from the time of admission according to the timeline. The dotted line can differ by size, color, and/or spacing. Administration of a therapy is indicated relative to the timeline by a shaded region. The start of the region is the start of the therapy such as a bolus. Alternatively, medication such as orally administered medication can be indicated with a differently coded line. The shading can indicate the duration or effective period of the medication. The assessment unit can assess the effectiveness of the administered therapy after a given period has elapsed and provide a recommendation such as an alternative therapy if the administered therapy is ineffective.

The subject vital signs 100 are graphically plotted relative to the timeline which include RR 102 in breaths per minute, HR 104 in beats per minute (bpm), BP 106 of systolic and diastolic in mmHg, and SpO2 108 as a percentage. The units of measure are shown with the scale for each vital sign indicated on the vertical axis. The values plotted are the measured values which use less space and provide a trajectory of the patient. The healthcare practitioner can obtain the vital sign waveforms by selecting the vital sign, e.g. clicking on the vital sign with a mouse or other input device.

The SOFA scoring 110 includes color coded icons 112 for each body organ system scored. The color coded icons are illustrated as a stack of rectangles. The box is clear if not scored based on insufficient information available. As information becomes available either through vital signs, lab results, or patient history retrieval boxes can be coded such as green for functioning, yellow for as risk, and red for failing. The SOFA scores determined by the patient assessment unit 24 include hepatic, renal, coagulation, respiratory, and cardiovascular body organ systems. In the illustrated example, based on the measured vital signs at the initial contact/triage assessment the initial SOFA score 114 respiratory is in the yellow zone, e.g. shortness of breath and respiration rate, and cardiovascular is in the green zone, e.g. HR, BP, SpO2 within acceptable ranges. At time t=2 hours 20 min, RR and HR increases and the patient assessment unit 24 determines the SOFA respiratory is failing and the cardiovascular is at risk. The patient watch-dog unit 42 constructs a display which includes the changed SOFA score 116 with cardiovascular coded yellow and respiratory coded red. The changed scores are added to the constructed display placed according to the timeline with the original scoring icons included to provide the seamless picture of the patient trajectory. Each scoring system with symbols or icons and vital signs in the constructed display contribute to provide information succinctly about different perspectives of the patient trajectory.

A change in care type event indicator 118 is shown as a bold color coded vertical dotted line. The indicator is placed according to the timeline which represents to admission of the subject from the waiting area to the ER.

Additional information can be obtained by the healthcare practitioner using the patient history popup window 82 by selecting the patient history button or icon in the upper right corner. The patient history popup windows list chronologically relevant patient history. The relevant patient history includes active diagnosis with related medications, historical vital signs, and lab panels performed. The lab panels can be further investigated by selecting the icon for the lab panel which provides another popup window with the lab panel values or results.

The deterioration detail and recommendation popup window 84 is invoked using the combination scoring icon 94. The deterioration detail 120 includes a date/time timestamp of the changed score and the values of the changed score, and a summary of the vital signs which caused the change in scoring. The changed score is express visually with the color coded icons, e.g. green coded circle labeled 4 with an arrow to a yellow coded circle labeled 3. The vital sign which caused the change in scoring are expressed symbolically and succinctly with color coding and the value changes for each vital signs. Respiration rate is expressed succinctly as “RR: 16->22 bpm” and the expression color coded red. Heart rate is expressed succinctly as “HR: 80->108 bpm” and the expression color coded yellow.

The recommendation 122 or recommendation popup window can include recommendations directed to the healthcare practitioner in a role. For example, the healthcare practitioner can include individuals in a variety of roles such as triage nurse, doctor, ED nurse, technician, etc. The recommendation displayed is for a nurse role which includes alerting the attending physician, and to work with staff to get a bed prepared for the patient. The recommendations displayed can be specific to the role of the healthcare practitioner performing the inquiry or can include all roles.

With graphical plots and scoring indicators placed according to a timeline, the maximum amount of information can be communicated in a multi-lingual environment. The constructed display includes line graphs of each monitored vital sign, event indicators such as color coded dotted lines, and scoring indicators such as color coded icons. The display for each monitored subject organizes by care type and location, the indicator of the triage or SOFA score. The display includes a timeline measured from the point of initial contact and the monitored vital signs, the event indicators, and the scoring indicators graphically displayed according to the timeline. With the displayed trajectory, healthcare practitioners can quickly determine a course of action. By more quickly determining a course of action and assessing changes, patient flow through the ED can be increased. With an increase in patient flow, wait time can be reduced.

With reference to FIG. 4 one method of using an embodiment of the patient watch-dog and intervention/event timeline is flowcharted. In a step 129 a subject such as an arrival at an emergency department waiting area is setup by a triage clinician during the initial triage assessment or initial point of contact. The setup can include attaching a wearable patient monitoring device 12 to each subject as they arrive. The setup can include recording from the subject, the subject gender, age, and symptoms.

In a step 130, vital signs of a plurality of subjects are continuously monitored and transmitted by the monitoring devices 12, and each subject is monitored from the point of initial contact with the healthcare professional. The vital signs include blood pressure (BP), blood oxygen (SpO2), heart rate (HR), and respiratory rate (RR). The transmitted vital signs are received by the patient assessment unit 24.

The triage score for each subject is determined by the patient assessment unit 24 based on the received vital signs, subject gender, subject age, and subject symptoms in a step 132. The SOFA scores are determined based on the information received which can include the vital signs, lab results, and/or patient history.

In a decision step 134, a change in score is evaluated. The change in score can be the determined triage score and/or the determined SOFA score. If a score is changed, then an event is generated based on a change in the determined score by the event unit 32 in a step 136. The event can be a deterioration event. The event can be an improvement event such as an improvement in the subject condition, or discharge consideration. The step can include modifying the frequency of the transmission of vital signs by the portable subject monitors 12. The step can include sending a notice such as an alert and/or advisory to one or more devices utilized by the healthcare practitioners.

In a step 138, a received administered therapy by a subject is recorded from the transmission of a therapy delivery device 30 or by entry of a healthcare practitioner. If a subject receives an administered therapy, then in a step 140 an administered therapy event is generated. The event occurs when the therapy is actually administered, e.g. fulfilling an ordered medication. The therapy event can include sending a notice such as an alert and/or advisory to one or more devices utilized by the healthcare practitioners. The sending of alerts and/or advisories can be limited by individual preferences, site policies, industry practices, and the like.

A change in care type is received in a step 142. The change in care type can be received by entry of a healthcare practitioner and/or a change in location of the subject such as recording the entry to the ED and/or a ED bed location as received by the patient tracking unit 40. If care type of a subject changes, then a care type change event is generated in a step 144.

In a step 146, an intervention is determined based on current scoring and/or administered therapies. The step can include making an assessment of the effectiveness of an administered therapy. For example, a therapy of administration of a saline fluid based on the current scoring and symptoms can be recommended. In another example, a predetermined interval since the administration of a therapy has elapsed and no change in vital signs or scoring has occurred, but another recommendation can be made. The recommendation is made in a step 148. The recommendation can be further refined to separate recommended steps by healthcare role. The step can include sending a notice such as an alert and/or advisory which can be directed based on healthcare role.

The dashboard display for the corresponding subject is constructed/reconstructed in a step 150. The constructed dashboard display of a patient trajectory for each subject includes the monitored vital signs, the determined triage score, and the subject gender, the subject age, and the subject symptoms. The constructed dashboard display includes an indicator for each generated event according to a timeline from the point of initial contact and/or initial assessment. The patient trajectory can include an indicator of the administered therapy according to the timeline. The step can include constructing a dashboard display for each subject and/or constructing a composite display for a plurality of subjects. The step can include constructing/reconstructing a care type status or a waiting room and ED display as described in reference to FIG. 2. In a step 152 the constructed displays are displayed on one or more display devices 44.

The method is continued in a step 154. The method repeats continuously until a termination action. The method includes the monitoring of one or more subjects from the point of initial contact and/or initial triage assessment. The method continues as each subject transitions from one care type unit to another. As the subject transitions to special care type unit such as a CCU or ICU, the monitoring can continue, and scoring systems exchanged according to the unit care type. A non-transitory computer-readable storage medium carrying software which controls one or more electronic data processing devices to perform the method.

It is to be appreciated that in connection with the particular illustrative embodiments presented herein certain structural and/or function features are described as being incorporated in defined elements and/or components. However, it is contemplated that these features may, to the same or similar benefit, also likewise be incorporated in other elements and/or components where appropriate. It is also to be appreciated that different aspects of the exemplary embodiments may be selectively employed as appropriate to achieve other alternate embodiments suited for desired applications, the other alternate embodiments thereby realizing the respective advantages of the aspects incorporated therein.

It is also to be appreciated that particular elements or components described herein may have their functionality suitably implemented via hardware, software, firmware or a combination thereof. Additionally, it is to be appreciated that certain elements described herein as incorporated together may under suitable circumstances be stand-alone elements or otherwise divided. Similarly, a plurality of particular functions described as being carried out by one particular element may be carried out by a plurality of distinct elements acting independently to carry out individual functions, or certain individual functions may be split-up and carried out by a plurality of distinct elements acting in concert. Alternately, some elements or components otherwise described and/or shown herein as distinct from one another may be physically or functionally combined where appropriate.

In short, the present specification has been set forth with reference to preferred embodiments. Obviously, modifications and alterations will occur to others upon reading and understanding the present specification. It is intended that the invention be construed as including all such modifications and alterations insofar as they come within the scope of the appended claims or the equivalents thereof. That is to say, it will be appreciated that various of the above-disclosed and other features and functions, or alternatives thereof, may be desirably combined into many other different systems or applications, and also that various presently unforeseen or unanticipated alternatives, modifications, variations or improvements therein may be subsequently made by those skilled in the art which are similarly intended to be encompassed by the following claims.

1. A medical system, comprising:
a plurality of portable vital sign monitors, each monitor continuously monitors vital signs of a subject from a point of initial contact with a healthcare professional and wirelessly transmits the monitored vital signs, and the vital signs include blood pressure (BP), blood oxygen (SpO2), heart rate (HR), and respiratory rate (RR);
a patient assessment unit which receives the transmitted monitored vital signs of each subject and determines a triage score based on the received vital signs, and subject gender, subject age, and subject symptoms;
a patient watch-dog unit which constructs a display of a patient trajectory of each subject and the trajectory includes the monitored vital signs, the determined triage score, and the subject gender, the subject age, and the subject symptoms; and
at least one display device which display the constructed patient trajectory of at least one subject.

2. The medical system according to claim 1,
wherein the patient watch-dog unit constructs a care status display of icons organized by care type which includes at least one of a waiting area and an emergency department, and each icon represents one subject; and
wherein the at least one display device displays the constructed care status display.

3. The medical system according to claim 2, wherein the care status display includes both the emergency department and the waiting area for the emergency room and the icons in the waiting room are color-coded in accordance with triage score.

4. The medical system according to claim 2, wherein the patient assessment unit further determines at least one sequential organ failure assessment (SOFA) score based on received lab results and the monitored vital signs;
wherein the icons further include an indicator of at least one of the determined triage score in the waiting area, and the at least one determined SOFA score in the emergency department.

5. The medical system according to claim 1, wherein the display of the patient trajectory includes a timeline measured from the point of initial contact and the monitored vital signs graphically displayed relative to the timeline.

6. The medical system according to claim 4, further including:
an event unit which generates an event based on at least one of:
a change in either one of the determined triage score and the at least one determined SOFA score;
administration of a therapy to at least one subject; and
a change in care type; and
wherein the constructed display of the trajectory includes an indicator of the event according to the timeline.

7. The medical system according to claim 6, wherein at least one event generated by the event unit includes a recommendation for a therapy; and
wherein the indicator of the at least one event includes a change indicative of an included recommendation.

8. The medical system according to claim 6, wherein at least one event generated by the event unit includes a plurality of notices each directed to a healthcare practitioner with recommendations based on a healthcare role.

9. The medical system according to claim 6, further including:
wherein the patient assessment unit assesses the effectiveness of the administered therapy and generates a recommendation for an alternative therapy; and
wherein the display of the patient trajectory includes an indicator of the recommended alternative therapy according to the timeline.

10. (canceled)

11. (canceled)

12. A method of monitoring patients, comprising:
receiving continuously monitored and transmitted vital signs of a plurality of subjects, each subject monitored from a point of initial contact with a healthcare professional and the vital signs include blood pressure (BP), blood oxygen (SpO2), heart rate (HR), and respiratory rate (RR);
determining a triage score for each subject based on the received vital signs, subject gender, subject age, and subject symptoms;
constructing a display of a patient trajectory for each subject and the trajectory includes the monitored vital signs, the determined triage score, and the subject gender, the subject age, and the subject symptoms;
displaying the constructed patient trajectory of at least one subject on a display device.

13. The method according to claim 12, further including:
attaching a wearable patient monitor to each of the plurality of subjects at the point of initial contact upon entering a waiting area for an emergency department of a first triage assessment by a triage clinician and the wearable patient monitor continuously monitors and transmits vital signs; and
recording from the subject the subject gender, the subject age, and the subject symptoms during the first triage assessment; and
constructing a care status display of monitored subjects organized by care type which include the waiting area and the emergency department, and each subject in the waiting area is indicated with a color coded icon indicative of the determined triage score; and
displaying the care status display.

14. (canceled)

15. (canceled)

16. The method according to claim 12, further including:
generating an event based on a change in the determined triage score; and
wherein constructing the patient trajectory includes indicating each determined triage score and the generated event according to the timeline.

17. The method according to claim 12, further including:
identifying with the care status display at least one subject to change to the emergency department care type from the waiting area care type based on a most acute of the indicated triage score;
identifying with care status display a care type location in the emergency department with either one of empty location or a least acute sequential organ failure assessment (SOFA) score subject in the location;
admitting the identified at least one subject to change to the emergency department care type to the identified care type location in the emergency department;
generating an event in response to a change in care type; and
notifying the responsible attending physician of the admitted subject to the identified emergency department care location;
wherein constructing the patient trajectory includes indicating the change in care type according to the timeline and constructing the care type status includes changing the icon representing the admitted at least one subject from the waiting area to the emergency department.

18. The method according to claim 12, further including:
recording an administered therapy to a subject at the time of administration based on an ordered therapy;
generating an event for the administered therapy;
assessing the effectiveness of the administered therapy based on a predefined interval and the monitored vital signs;
notifying at least one healthcare practitioner of the assessed effectiveness of the administered therapy at a conclusion of the predetermined interval; and
wherein constructing the patient trajectory including indicating the administered therapy according to the timeline.

19. A non-transitory computer-readable storage medium carrying software which controls one or more electronic data processing devices to perform the method according to claim 1.

20. (canceled)
